(19) United States
(12) Patent Application Publication
(10) Pub. No.: US 2015/0112303 A1
(43) Pub. Date: Apr. 23, 2015

(54) Method and Device for Blood Vessel Cannulation

(71) Applicant: Edward Lazzarin, Coral Gables, FL (US)

(72) Inventor: Edward Lazzarin, Coral Gables, FL (US)

(21) Appl. No.: 14/335,684

(22) Filed: Jul. 18, 2014

Related U.S. Application Data
(60) Provisional application No. 61/847,695, filed on Jul. 18, 2013.

(51) Int. Cl. A61M 5/42

(52) U.S. Cl. CPC A61M 5/425
USPC 604/507; 604/115

(57) ABSTRACT

A device and method secure a blood vessel for cannulation. The device includes two parallel skids. The device is placed on the skin of a patient with a skid on each side of a blood vessel to be phlebotomized. When the skids are pressed on the skin, fluid in the tissue is displaced to the space between the skids. The blood vessel becomes secured and unable to move or roll. With the skids still pressed on the skin, the cannula can be inserted into the blood vessel. The width and spacing of the skids are adjusted to maximize the securing effect on the blood vessel.

### CROSS-REFERENCE TO RELATED APPLICATIONS

This application claims the benefit of U.S. Provisional Application No. 61/847,695, filed Jul. 18, 2013, which is hereby incorporated by reference.

### STATEMENT REGARDING FEDERALLY SPONSORED RESEARCH OR DEVELOPMENT

Not Applicable

### THE NAMES OF PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

The invention relates to devices and methods for phlebotomy and hemodialysis.

2. Description of the Related Art

Phlebotomy is the process of making an incision in a vein. Typically, a cannula tip of a catheter is inserted into a blood vessel. The catheter may need to be inserted for collecting a blood sample for a patient, for connecting a patient to a dialysis machine, or for injecting fluids intravenously.

The state of the art uses unassisted, unguided phlebotomy. Frequently, repeated attempts to insert a catheter into a vein are required. Each attempt can cause painful, swollen hematomas. As a result, the patients become fearful and irritated.

The difficulty with inserting catheters involves not only locating the vein but also issues with anatomy. Blood vessels are generally found in loose connective tissue, under the skin, and over muscles and tendons. Blood vessels are not anchored to other tissue. Accordingly, blood vessels easily can move in relation to surrounding tissue. Approaching catheters frequently will push a vein away in an opposite direction. As the vein is deflected, the catheter can miss or injure the targeted vessel.

FIGS. 12-14 illustrate the problem of vein deflection that occurs in the prior art. FIG. 12 shows a vein 40. A catheter 50 includes a distal cannula 51 and a proximal connector 52. As the catheter 50 approaches the vein 40, the vein 40 can roll to the side as shown in FIG. 13. If the phlebotomist continues to insert the catheter 50, the catheter 50 can injure the vein by penetrating the opposite wall of the vein 40 as shown in FIG. 14.

### BRIEF SUMMARY OF THE INVENTION

An object of the invention is to provide a method and device for blood vessel cannulation that overcome the disadvantages of the devices and methods of this general type and of the prior art.

With the foregoing and other objects in view there is provided, in accordance with the invention, a device for stabilizing a blood vessel for cannulation. The device includes a first skid, a second skid, and a connector. The first skid has a bottom surface. The second skid has a bottom surface and runs parallel and adjacent to the first skid. The connector interconnects the first skid and the second skid. The connector has a bottom surface at least partially above a notional line drawn between said bottom surface of said first skid and said bottom surface of said second skid to define a channel under the connector. The channel runs parallel to the skids. The skids are placed on the skin. The skids are arranged by the phlebotomist to be located on opposing sides of a targeted blood vessel. The skids are spaced slightly wider than the blood vessel. The skids are pressed against the skin. As the skids are pressed, the blood vessel becomes held. The phlebotomist can insert a catheter without fear of the blood vessel moving during insertion. The channel in the connector provides clearance for the blood vessel. The channel prevents blood flow through the blood vessel from being restricted when the skids are pressed against the skin.

In accordance with the objects of the invention, an apex of the bottom (i.e. inferior) surface of the connector can be spaced at least as high as a thickness of the blood vessel to be cannulized. The apex provides sufficient clearance for the blood vessel.

To provide sufficient room for the blood vessel, the first skid and the second skid can be separated by a distance greater than a diameter the blood vessel to be cannulized. Based on average anatomy, the distance separating the first skid from the second skid should be at least five millimeters (>5 mm). A device where the first skid and the second skid are separated by at least nine millimeters (>9 mm) has been shown be effective. A device where the distance separating the first skid from the second skid is no more than fifteen millimeters can be effective.

The device can include a handle connected to the connector. The handle can be used to provide force to device to press the skids against the skin. The handle can be placed at an obtuse angle to the channel. An obtuse angle allows a patient to phlebotomize him or herself with his or her other hand. The handle can be inclined at an obtuse angle from a plane defined by the forks. The incline provides clearance from the patient's body when the phlebotomist applies pressure with the device. The lateral angle and the incline angle both can be included in one device.

The ends of the skids can be rounded and beveled. Rounding the ends of the skids makes the skids less likely to injure a patient.

The device and the skids in particular can be made of resilient medical grade materials. Examples of suitable materials include steel, aluminum, and polymer resins.

Having the skids be at least five millimeters wide provides suitable displacement on surrounding tissue to secure the blood vessel when the skids are pressed on the surrounding skin. For an even more effective device, the skids should each be at least seven millimeters wide. When the skids are too narrow, the displacement can be insufficient to hold the blood vessel.

In accordance with a further object of the invention, a method of stabilizing a blood vessel for canalization is described. The first step involves applying topical force to skin on opposite sides of a blood vessel. The force should be applied alongside the length of the blood vessel into which the catheter will be inserted. Once the blood vessel is held, the next step is inserting a cannula of a catheter into the blood vessel. Applying the force two to five millimeters laterally from the blood vessel provides enough displacement to secures the blood vessel. A further consideration is to avoid restricting blood flow through the blood vessel while securing the blood vessel for cannulation.

Other features that are considered as characteristic for the invention are set forth in the appended claims.

Although the invention is illustrated and described herein as embodied in a method and a device for blood vessel cannulation, the invention should not be limited to the details shown in those embodiments because various modifications and structural changes may be made without departing from the spirit of the invention while remaining within the scope and range of equivalents of the claims.

The construction and method of operation of the invention and additional objects and advantages of the invention is best understood from the following description of specific embodiments when read in connection with the accompanying drawings.

### BRIEF DESCRIPTION OF THE SEVERAL VIEWS OF THE DRAWING

FIGS. 1-11 are canceled.

FIG. 12 is a diagrammatic top side view of an approaching cannula according to a prior-art cannulation.

FIG. 13 is a diagrammatic top side view of a blood vessel rolling according to the prior-art cannulation.

FIG. 14 is a diagrammatic top side view of a blood vessel being injured according to the prior-art cannulation.

FIG. 15 is a sectional view of blood vessel according to the prior art.

FIG. 16A is a diagrammatic top side view of the device according to the invention.

FIG. 16B is a bottom side view of the device shown in FIG. 16A.

FIG. 17 is a top side view of the device being held by a user's left hand.

FIG. 18 is a top side view of the device being held by a user's right hand.

FIG. 19 is a top side view of the device holding a blood vessel in preparation for being cannulized while being held by a user's left hand.

FIG. 20 is a top side view of the device holding a blood vessel in preparation for being cannulized while being held by a user's right hand.

FIG. 21 is a top side view of the device holding a blood vessel with the cannula inserted in the blood vessel.

FIG. 22 is a top side view of the device in relation to a strip of material from which the device can be stamped.

FIG. 23 is a front sectional view of a blood vessel held by the device.

FIG. 24 is a top view of a patient's arm with a blood vessel shown in phantom.

FIG. 25 is a top view of the patient's arm shown in FIG. 24 with the blood vessel being held by the device.

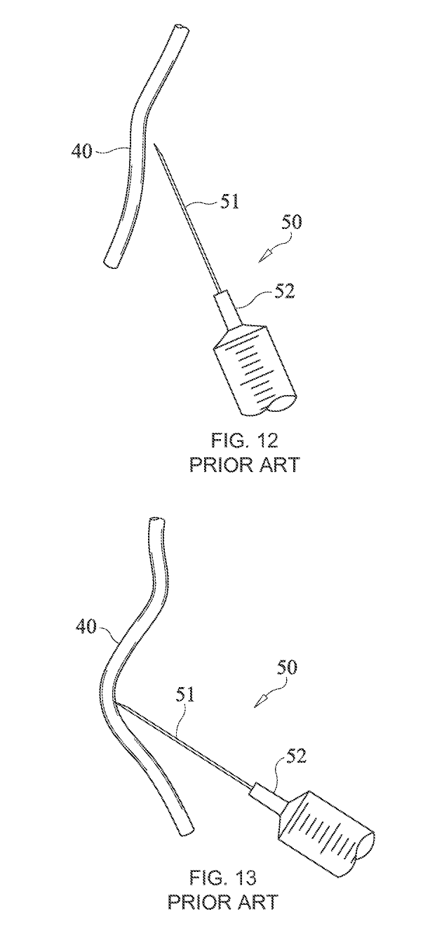

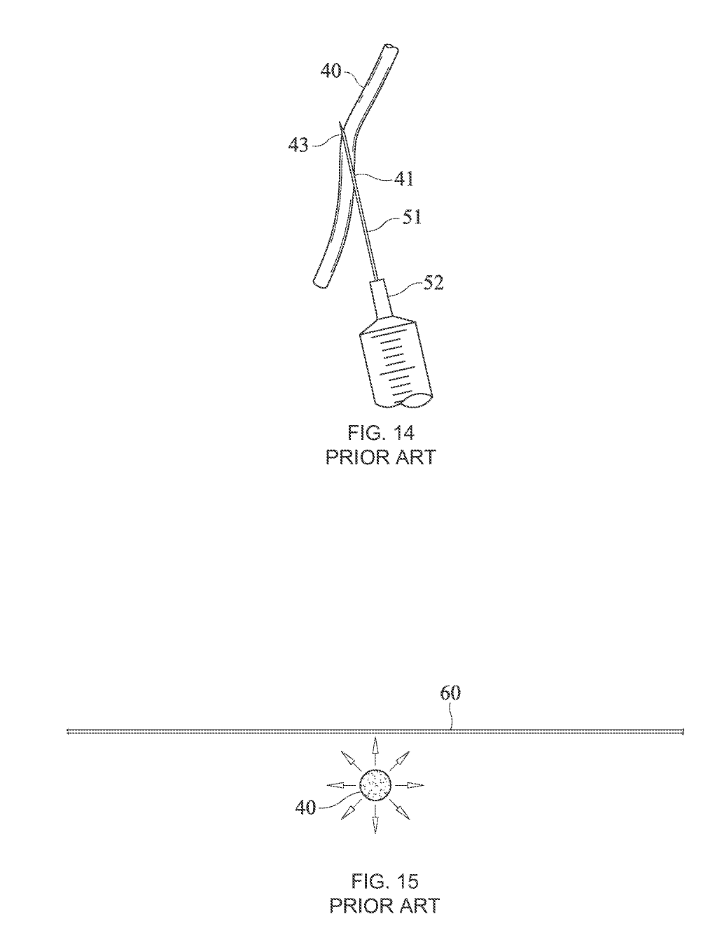

### DETAILED DESCRIPTION OF THE INVENTION

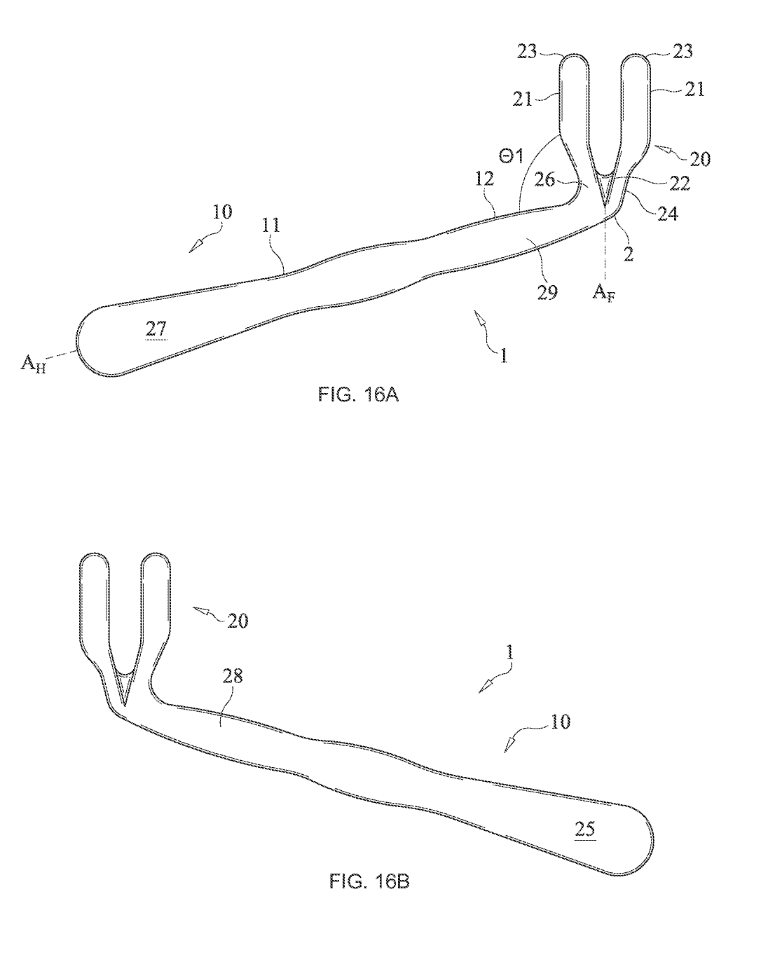

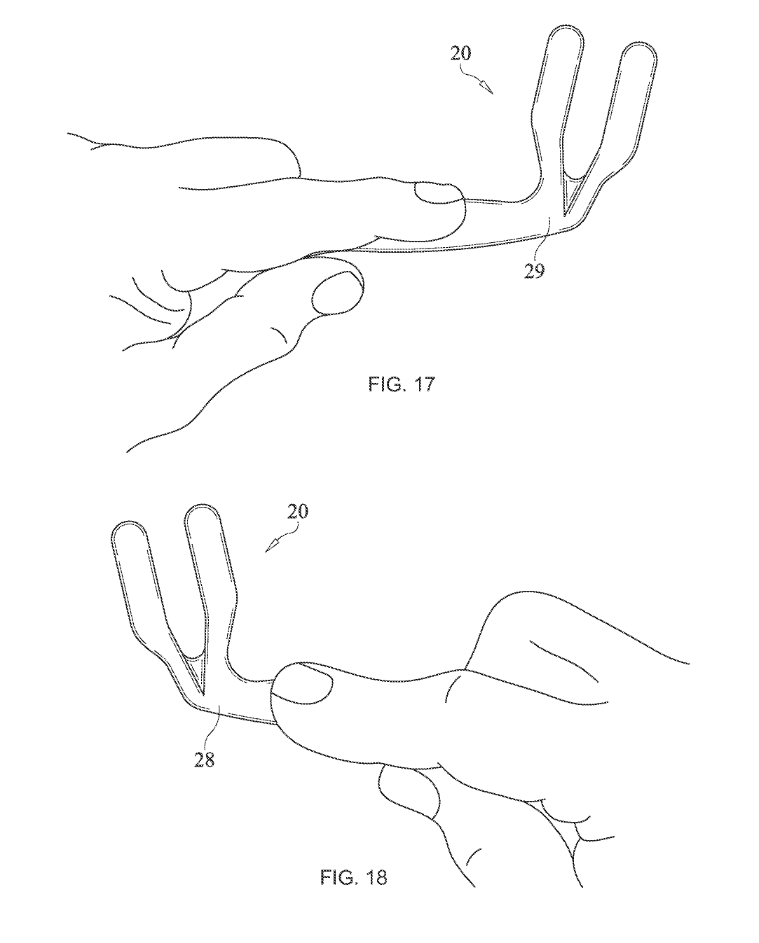

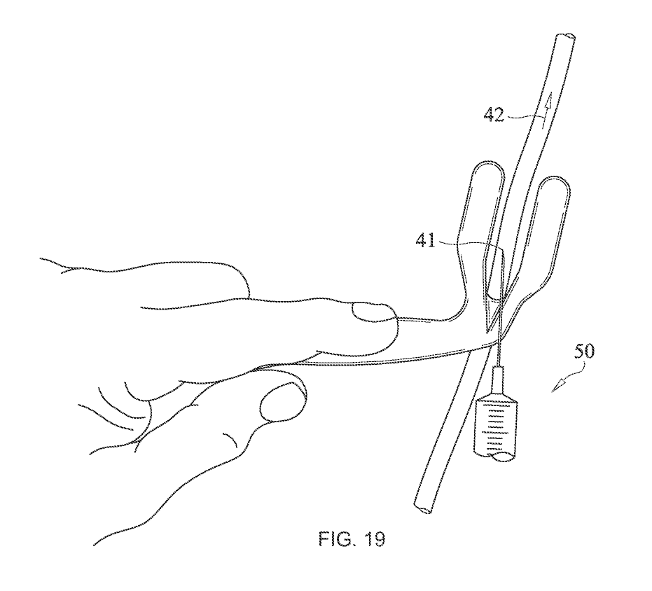

FIGS. 16A-16B show a first embodiment of phlebotomy tool 1 according to the invention. The tool 1 has a handle 10 that is coplanar with a distal fork 20.

As mentioned, the tool 1 includes a distal fork 20 and a proximal handle 10. The fork 20 is placed against the skin surface to fix an underlying blood vessel. The handle 10 is used to position the fork 20 and to apply pressure with the fork 20 against the skin 60 as shown in FIGS. 17 and 18.

The fork 20 includes two adjacent parallel skids 21. The skids 21 are spaced by a distance that is at least as wide as a width of a blood vessel that is to be cannulized. The distance between the skids is preferably nine millimeters (9 mm). This distance is based on average anatomy. Each skid 21 has a width that is preferably seven millimeters (7 mm). The width displaces enough volume of tissue to stabilize a blood vessel between skids 21 that are spaced by nine millimeters. The length of each skid is greater than the length of the portion of the cannula that is to be inserted within the blood vessel. Preferably, the length is seven millimeters (7 mm). The skids each have a thickness.

An inferior surface 25 of each skid 21 is preferably flat. The inferior surface 25 is used to contact the skin of the patient. The distal surface 25 displaces tissue to secure the blood vessel when the skids 21 are pressed on the skin 60.

In the embodiment shown in FIGS. 16A-16B, the superior surface 27 of each skid 21 is flat. By having the both surfaces 25 and 27 be flat, the tool 1 can be flipped over and used to fix a blood vessel in the opposite arm.

A distal end 23 of each skid 21 is rounded for safety. In particular, the transverse cross section of each skid 21 is semicircular.

A connector 26 interconnects the first skid 21 and the second skid. The connector 26 holds the skids 21 in relation to each other. The connector 26 preferable does not restrict blood flow through the blood vessel that is to be cannulized.

An inferior surface 28 of the connector 26 has a channel 22 that is concave and arcuate formed therein. The channel 22 in the inferior surface 28 of the connector 26 can be placed over the blood vessel and then when the skids 21 are depressed, blood flow through the blood vessel 40 is not restricted.

A superior surface 29 of the connector is concave and arcuate. The superior surface 29 of the connector 26 can be placed over the blood vessel 40 when the tool 1 is inverted for use on a blood vessel in an opposing arm. When the skids 21 are depressed, blood flow through the blood vessel 40 is not restricted by the connector 26.

A distal stem 24 is connected to the distal stem 24. The distal stem 24 acts as a lever arm for applying torque to the fork 20.

An angle Θ₁ is defined between the axis A_{F} of the fork 20 and the axis A_{H} of the handle 1. The angle Θ₁ is preferably twenty degrees (20°). The angle Θ₁ is can be preconfigured to allow the patient to use the device on his or herself. The angle Θ₁ can be preconfigured for use by a practitioner.

The handle 10 of the tool 1 is used to hold the tool 1 and to apply force to the fork 20. Bumps 11 are added to the handle 10 to make holding the handle 10 more secure.

The tool 1 has a thickness of twenty millimeters (20 mm). The tool 1 is preferably made of stainless steel, aluminum, or a polymer.

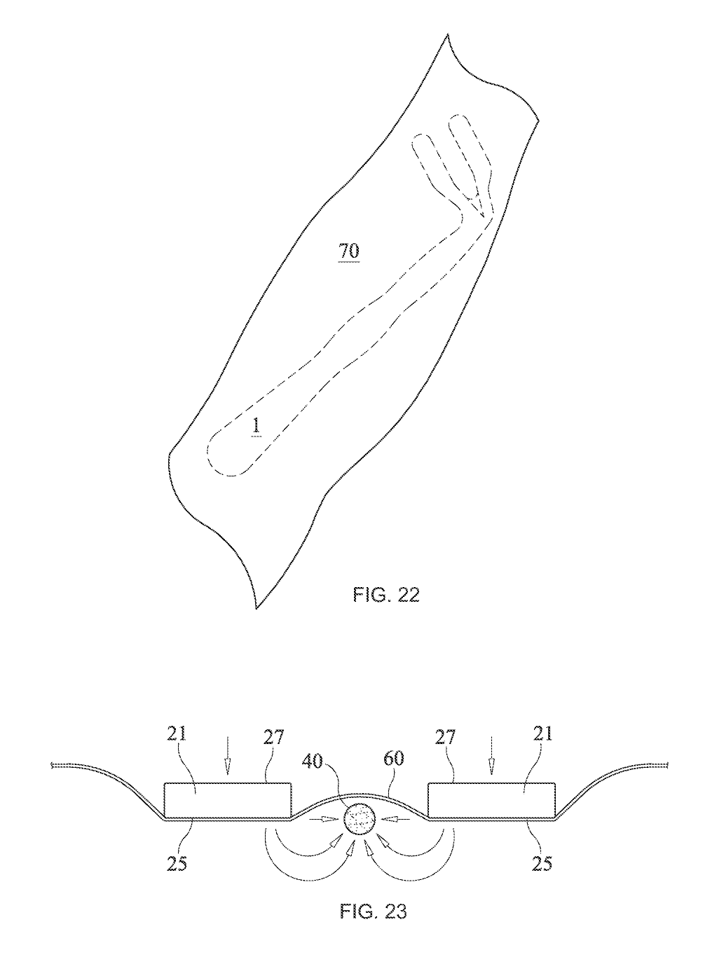

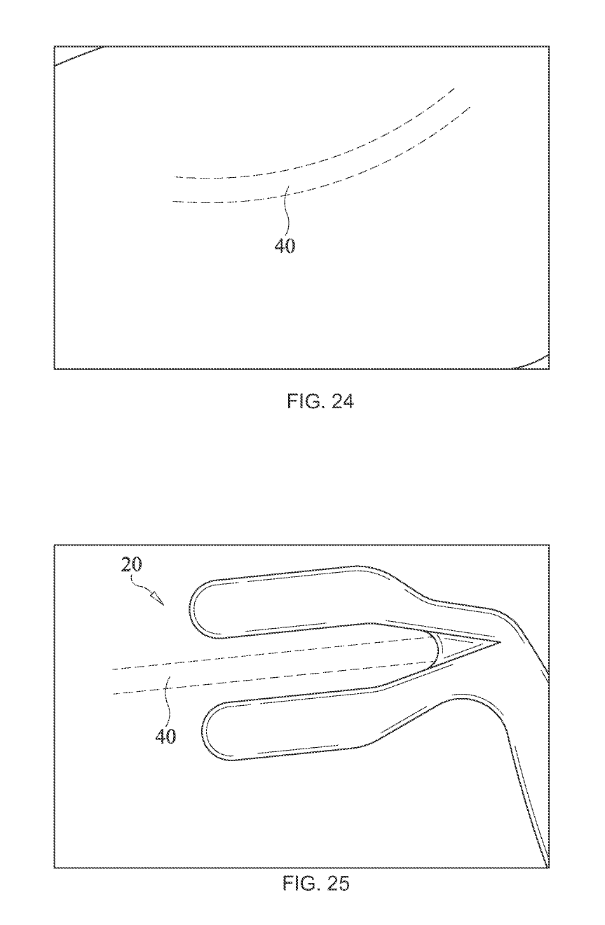

The tool 1 can be made according to the method shown in FIG. 22. The first step is to provide a sheet of material. A preferred embodiment of a sheet is a fifty millimeter (50 mm) wide fourteen gauge stainless steel sheet. Next, the tool shape is stamped from the sheet as shown in FIG. 22. A set of devices can be stamped from a given sheet of steel to maximize the number of devices that can be produced from the sheet of raw material.

FIGS. 19-21 show a preferred method for stabilizing a blood vessel for canalization using the tool 1. In FIG. 19, the fork 20 is placed on the skin of patient. The fork 20 is moved so that the forks 20 are located on either side of the blood vessel. The skids 21 should be aligned to be as parallel to the blood vessel as the patient's anatomy will allow. As shown in FIG. 23, downward force is applied by the skids 21 onto the skin of the patient. As long as the downward force is being applied, an opposing hydraulic force will cause the blood vessel 40 to be held between the skids 21.

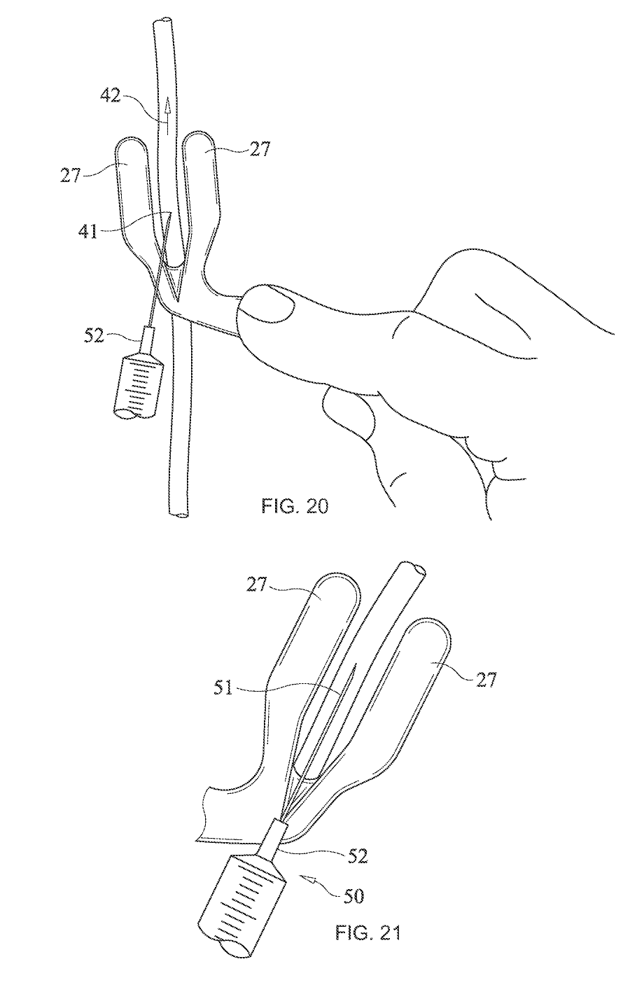

FIG. 20 shows how a preferred method for inserting a catheter in the blood vessel 40. An entry 40 of the catheter is located relatively close to the connector 26. The catheter 50 includes a distal cannula 51 and proximal connector 52. The catheter 50 is inserted through the skin of the patient at an angle into the blood vessel 40. The catheter 50 is inserted in a direction 42 that is generally distal into the blood vessel 27. FIG. 21 shows the catheter in an inserted position.

What is claimed is:

1. A device for stabilizing a blood vessel for cannulation comprising:
a first skid having a bottom surface;
a second skid having a bottom surface and running parallel and adjacent to said first skid; and
a connector interconnecting said first skid and said second skid, said connector having a bottom surface at least partially above a notional line drawn between said bottom surface of said first skid and said bottom surface of said second skid to define a channel under said connector, said channel being parallel to said skids.

2. The device according to claim 1, wherein an apex of said bottom surface of said connector is at least as high as a thickness of the blood vessel to be cannulized.

3. The device according to claim 1, wherein said first skid and said second skid are separated by a distance greater than a diameter the blood vessel to be cannulized.

4. The device according to claim 1, wherein said distance separating said first skid from said second skid is at least five millimeters.

5. The device according to claim 3, wherein said distance separating said first skid from said second skid is at least nine millimeters.

6. The device according to claim 1, wherein said distance separating said first skid from said second skid is no more than 15 millimeters.

7. The device according to claim 1, further comprising a handle connected to said connector.

8. The device according to claim 7, wherein said handle is at an obtuse angle to said channel.

9. The device according to claim 1, wherein said handle is inclined at an obtuse angle from a plane defined by said first skid and said second skid.

10. The device according to claim 1, wherein at least one of said first skid and said second skid has a rounded distal end.

11. The device according to claim 1, wherein said skids are formed from steel.

12. The device according to claim 1, wherein said skids are formed from aluminum.

13. The device according to claim 1, wherein said skids are formed from a polymer.

14. The device according to claim 1, wherein said first skid and said second skid each are at least five millimeters wide.

15. The device according to claim 14, wherein said first skid and said second skid are each at least seven millimeters wide.

16. A method of stabilizing a blood vessel for canalization, which comprises:
applying topical force to skin on a first side of the blood vessel while applying topical force to skin on a second side of the blood vessel, said first side and said second side opposing each other; and
inserting a cannula into the blood vessel.

17. The method according to claim 16, which further comprises applying the force along the sides of the blood vessel for at least a length of insertion of the cannula.

18. The method according to claim 16, which further comprises applying the force two to five millimeters laterally from the blood vessel.

19. The method according to claim 16, which further comprises not restricting blood flow through the blood vessel.
